(19) United States
(12) Patent Application Publication
(10) Pub. No.: US 2011/0319743 A1
(43) Pub. Date: Dec. 29, 2011

(54) ULTRASONIC PHOTOACOUSTIC IMAGING APPARATUS AND OPERATION METHOD OF THE SAME

(75) Inventor: Yoshiaki SATOH, Ashigarakami-gun (JP)

(73) Assignee: FUJIFILM CORPORATION, Tokyo (JP)

(21) Appl. No.: 13/167,352

(22) Filed: Jun. 23, 2011

(30) Foreign Application Priority Data
Jun. 24, 2010 (JP) 2010-143406

(51) Int. Cl. A61B 6/00

(52) U.S. Cl. 600/407

(57) ABSTRACT

An ultrasonic photoacoustic imaging apparatus which includes a probe incorporating an array transducer having a plurality of transducers and an acoustic image generation unit that generates, based on a mixed signal obtained by converting an acoustic wave, in which an ultrasonic wave and a photoacoustic wave are mixed and making use of a difference between phase shift aspect of electrical signals of the ultrasonic waves from the same reflection source in the inside of the subject and phase shift aspect of electrical signals of the photoacoustic waves from the same generation source in the inside of the subject, an electrical signal reflecting the ultrasonic wave and an electrical signal reflecting the photoacoustic wave, and generates an ultrasonic image based on the electrical signal reflecting the ultrasonic wave and a photoacoustic image based on the electrical signal reflecting the photoacoustic wave.

### BACKGROUND OF THE INVENTION

1. Field of the Invention

The present invention relates to an ultrasonic photoacoustic imaging apparatus for generating an ultrasonic image by projecting an ultrasonic wave into an inside of a subject and detecting a ultrasonic wave reflected from the inside of the subject and generating a photoacoustic image by projecting light into the inside of a subject and detecting a photoacoustic wave generated in the inside of the subject. The invention also relates to an operation method of the same.

2. Description of the Related Art

As one of the methods of obtaining an internal tomographic image of a subject, an ultrasonic imaging is known in which an ultrasonic image is generated by projecting an ultrasonic wave into an inside of a subject and detecting an ultrasonic wave reflected from the inside of the subject, thereby obtaining a morphological internal tomographic image of the subject. In the mean time, development of devices for displaying not only a morphological tomographic image but also a functional tomographic image has been in progress in the field of subject examination. As one of such devices, a device that uses photoacoustic spectroscopy is known. In the photoacoustic spectroscopy, light having a predetermined wavelength (e.g., visible light, near infrared light, or intermediate infrared light) is projected into a subject and a photoacoustic wave which is an elastic wave generated in the inside of the subject as a result of absorption of the light energy by a particular substance is detected to quantitatively measure the density of the particular substance. The particular substance in an inside of a subject is, for example, glucose, hemoglobin, or the like in blood. The technology of detecting photoacoustic wave and generating a photoacoustic image based on the detected signal in the manner described above is called photoacoustic imaging (photoacoustic tomography).

Development of ultrasonic photoacoustic imaging apparatus for obtaining an ultrasonic image and a photoacoustic image of an inside of a subject by applying these imaging methods and further obtaining a combined image by superimposing these tomographic images on top of each other with each image being identified by color has been in progress in recent years as described, for example, in Japanese Unexamined Patent Publication Nos. 2005-021380 and 2010-022816 and Japanese Patent Application Publication No. 2010-509977.

When trying to obtain an ultrasonic image by ultrasonic imaging and a photoacoustic image by photoacoustic imaging, tomographic image data are generally collected alternately with respect to each scanning (data of one line of the tomographic image) or each frame (data of one tomographic image) as described, for example, in Japanese Unexamined Patent Publication Nos. 2005-021380 (e.g., paragraph [0149]) and 2010-022816 (paragraphs [0040] to [0042]), Japanese Application Publication No. 2010-509977 (e.g., paragraphs [0042] and [0043]). The reason for this is that the ultrasonic wave and photoacoustic wave are identical in that they are acoustic waves propagating in the inside of a subject and this causes a problem that, when an acoustic wave is detected by a detector (probe or the like), it is simply difficult to determine whether the detected acoustic wave is an ultrasonic wave or a photoacoustic wave, or a problem that, when an ultrasonic wave and a photoacoustic wave are detected simultaneously by the same detector, these waves are detected as a superimposed single acoustic wave.

In order to cope with these problems, Japanese Unexamined Patent Publication No. 2005-021380 describes (in paragraphs [0153] to [0155]) a method in which arrangement is made such that the frequency of an ultrasonic wave and the frequency of a photoacoustic wave differ from each other, then the ultrasonic wave and photoacoustic wave are detected simultaneously by different detectors adapted to the respective frequencies, and the acoustic waves are separated through signal processing based on the frequency difference. The method described in Japanese Unexamined Patent Publication No. 2005-021380 has the following advantages over the conventional technology: capable of independently generating a ultrasonic image and a photoacoustic image even though the ultrasonic wave and photoacoustic wave are detected simultaneously by different detectors; capable of preventing distortion and image quality degradation of a combined image by reducing the influence of subject motion and timing difference in data collection between the ultrasonic image and photoacoustic image, capable of improving image construction speed for a tomographic image, and the like.

The method described in Japanese Unexamined Patent Publication No. 2005-021380, however, is only applicable to a case in which the ultrasonic wave and photoacoustic wave have different frequencies and they are detected by different detectors, thereby requiring a special detector, such as a two-frequency probe or the like. In contrast to this, it is desirable that an ultrasonic image and a photoacoustic image can be generated even though the ultrasonic wave and photoacoustic wave are detected simultaneously by the same detector without depending on the frequency. Further, if an ultrasonic image and a photoacoustic image are generated in parallel using the same detector, image construction speed may be improved with a simple structure.

The present invention has been developed in view of the circumstances described above, and it is an object of the present invention to provide an ultrasonic photoacoustic imaging apparatus, which employs ultrasonic imaging and photoacoustic imaging, capable of independently generating an ultrasonic image and a photoacoustic image even though ultrasonic wave and photoacoustic wave are detected simultaneously and without depending on the frequencies of these waves. It is a further object of the present invention to provide an operation method of the same.

### SUMMARY OF THE INVENTION

An ultrasonic photoacoustic imaging apparatus of the present invention is an apparatus, including:

an ultrasonic wave projection unit for projecting an ultrasonic wave into an inside of a subject;

a light projection unit for projecting light into the inside of the subject;

a probe capable of detecting the ultrasonic wave reflected from the inside of the subject by the projection of the ultrasonic wave into the inside of the subject and converting the detected ultrasonic wave into an electrical signal, and capable of detecting a photoacoustic wave generated in the inside of the subject by the projection of the light into the inside of the subject and converting the detected photoacoustic wave into an electrical signal; and

an acoustic image generation unit for generating an ultrasonic image based on the electrical signal of the ultrasonic wave detected by the probe and/or a photoacoustic image based on the electrical signal of the photoacoustic wave detected by the probe, wherein:

the probe includes an array transducer having a plurality of transducers; and

the acoustic image generation unit is a unit capable of generating, based on a mixed signal obtained by converting an acoustic wave, in which an ultrasonic wave and a photoacoustic wave are mixed, detected by each transducer during a predetermined capturing period to an electrical signal, and making use of a difference between phase shift aspect of electrical signals of the ultrasonic waves from the same reflection source in the inside of the subject in the respective mixed signals and phase shift aspect of electrical signals of the photoacoustic waves from the same generation source in the inside of the subject in the respective mixed signals, an electrical signal reflecting the ultrasonic wave and an electrical signal reflecting the photoacoustic wave, and generating the ultrasonic image based on the electrical signal reflecting the ultrasonic wave and the photoacoustic image based on the electrical signal reflecting the photoacoustic wave.

The term “ultrasonic wave” as used herein refers to an acoustic wave (elastic wave) projected by the ultrasonic wave projection unit and a reflection wave of the acoustic wave. The term “photoacoustic wave” as used herein refers to an acoustic wave generated by a photoacoustic effect. Further, a wave propagating the inside of the subject is simply referred to as the “acoustic wave” which means to include both the ultrasonic wave and photoacoustic wave. The term “ultrasonic image” as used herein refers to a tomographic image generated by ultrasonic imaging and the term “photoacoustic image” as used herein refers to a tomographic image generated by photoacoustic imaging. When the simple term “tomographic image” is used herein, it includes both the ultrasonic image and photoacoustic image.

The term “predetermined capturing period” as used herein refers to a period in which a transducer is able to detect, as a detector, an acoustic wave and capture the detected wave as an electrical signal.

The term “in which an ultrasonic wave and a photoacoustic wave are mixed” as used herein refers to that both the ultrasonic wave and photoacoustic wave are included in an acoustic wave detected by one of the transducers during the capturing period. This includes the case in which the ultrasonic wave and photoacoustic wave are detected simultaneously as a superimposed acoustic wave and the case in which the ultrasonic wave and photoacoustic wave are detected in temporally separated to the extent distinguishable within the capturing period.

The term “a mixed signal” as used herein refers to an electrical signal of an acoustic image, in which an ultrasonic wave and a photoacoustic wave are mixed, converted by the probe.

The term “ultrasonic waves from the same reflection source” as used herein refers to, with respect to reflected ultrasonic waves, the tissue structures inside of the subject that have caused the reflections are substantially the same, and the term “photoacoustic waves from the same generation source” as used herein refers to, with respect to photoacoustic waves, the tissue structures inside of the subject that have caused the generations are substantially the same.

The term “an electrical signal reflecting the ultrasonic wave” as used herein refers to an electrical signal generated based on a plurality of mixed signals detected and generated by each of the plurality of transducers and representing the relationship between an intensity (amplitude) of the reflected ultrasonic wave and time (e.g., a time from the time when the ultrasonic wave is projected by the ultrasonic projection unit to the time when the ultrasonic wave reaches the probe). The term “an electrical signal reflecting the photoacoustic wave” as used herein refers to an electrical signal generated based on a plurality of mixed signals detected and generated by each of the plurality of transducers and representing the relationship between an intensity (amplitude) of the photoacoustic wave and time (e.g., a time from the time when the light is projected by the light projection unit to the time when the photoacoustic wave reaches the probe).

Preferably, in the ultrasonic photoacoustic imaging apparatus of the present invention, the acoustic image generation unit is a unit that generates the electrical signal reflecting the ultrasonic wave by performing a first addition process for adding a plurality of mixed signals using ultrasonic wave delay data and under the condition of matching the phase shift of the electrical signal of the ultrasonic wave, and generates the electrical signal reflecting the photoacoustic wave by performing a second addition process for adding a plurality of mixed signals using photoacoustic wave delay data and under the condition of matching the phase shift of the electrical signal of the photoacoustic wave.

The term “ultrasonic wave delay data” as used herein refers to an amount of delay given to the mixed signals for phase matching which is appropriate for matching phase shifts of ultrasonic waves from the same reflection source in the respective mixed signals and term “photoacoustic wave delay data” as used herein refers to an amount of delay given to the mixed signals for phase matching which is appropriate for matching phase shifts of photoacoustic waves from the same generation source in the respective mixed signals.

Preferably, the acoustic image generation unit is a unit that generates the electrical signal reflecting the ultrasonic wave by performing a first threshold process on an electrical signal generated by the first addition process for decreasing signal strength less than a predetermined threshold value and generates the electrical signal reflecting the photoacoustic wave by performing a second threshold process on an electrical signal generated by the second addition process for decreasing signal strength less than a predetermined threshold value.

Further, it is preferable that the ultrasonic wave projection unit is a unit that projects a collimated ultrasonic wave. In this case, it is preferable that the ultrasonic photoacoustic imaging apparatus further includes a timing control unit for performing control such that projection timing of the collimated ultrasonic wave and projection timing of the light are synchronized.

Still further, it is preferable that the acoustic image generation unit is a unit that generates the ultrasonic image and the photoacoustic image in parallel.

Further, it is preferable that the acoustic image generation unit is a unit that generates the combined image after matching scales of the ultrasonic image and the photoacoustic image. In this case, it is preferable that the acoustic image generation unit is a unit that generates the combined image after matching scales of the ultrasonic image and the photoacoustic image.

Preferably, the probe doubles as the ultrasonic wave projection unit.

Preferably, the apparatus allows selection between an ultrasonic mode in which only the ultrasonic image is generated and a photoacoustic mode in which only the photoacoustic is generated. In this case, it is preferable that the apparatus allows switching between projection and non-projection of the ultrasonic wave in the photoacoustic mode.

Preferably, the acoustic image generation unit is a unit that performs the first addition process on a plurality of mixed signals on which a first frequency analysis process has been performed and the second addition process on a plurality of the mixed signal on which a second frequency analysis process has been performed, the second frequency analysis process being different from the first frequency analysis process in condition.

Preferably, the acoustic image generation unit is a unit that performs a third frequency analysis process on an electrical signal generated by the first addition process and a fourth frequency analysis process on an electrical signal generated by the second addition process, the fourth frequency analysis process being different from the third frequency analysis process in condition.

An operation method of the ultrasonic photoacoustic imaging apparatus of the present invention is a method, including the steps of:

projecting an ultrasonic wave and light into an inside of a subject;

using a probe, detecting the ultrasonic wave reflected from the inside of the subject and converting the detected ultrasonic wave into an electrical signal, and detecting a photoacoustic wave generated in the inside of the subject and converting the detected photoacoustic wave into an electrical signal;

generating an ultrasonic image based on the electrical signal of the detected ultrasonic wave and/or a photoacoustic image based on the electrical signal of the detected photoacoustic wave, wherein:

the probe includes an array transducer having a plurality of transducers; and

based on a mixed signal obtained by converting an acoustic wave, in which an ultrasonic wave and a photoacoustic wave are mixed, detected by each transducer during a predetermined capturing period to an electrical signal, and making use of a difference between phase shift aspect of electrical signals of the ultrasonic waves from the same reflection source in the inside of the subject in the respective mixed signals and phase shift aspect of electrical signals of the photoacoustic waves from the same generation source in the inside of the subject in the respective mixed signals, an electrical signal reflecting the ultrasonic wave and an electrical signal reflecting the photoacoustic wave are generated; and

the ultrasonic image is generated based on the electrical signal reflecting the ultrasonic wave and the photoacoustic image is generated based on the electrical signal reflecting the photoacoustic wave.

In the operation method of the ultrasonic photoacoustic imaging apparatus of the present invention, it is preferable that the electrical signal reflecting the ultrasonic wave is generated by performing a first addition process for adding a plurality of mixed signals using ultrasonic wave delay data and under the condition of matching the phase shift of the electrical signal of the ultrasonic wave, and the electrical signal reflecting the photoacoustic wave is generated by performing a second addition process for adding a plurality of mixed signals using photoacoustic wave delay data and under the condition of matching the phase shift of the electrical signal of the photoacoustic wave.

In the case described above, it is preferable that the electrical signal reflecting the ultrasonic wave is generated by performing a first threshold process on an electrical signal generated by the first addition process for decreasing signal strength less than a predetermined threshold value; and the electrical signal reflecting the photoacoustic wave is generated by performing a second threshold process on an electrical signal generated by the second addition process for decreasing signal strength less than a predetermined threshold value.

In the operation method of the ultrasonic photoacoustic imaging apparatus of the present invention, it is preferable that the ultrasonic image and the photoacoustic image are generated in parallel.

Further, it is preferable that a collimated ultrasonic wave is projected. In this case, it is preferable that control is performed such that projection timing of the collimated ultrasonic wave and projection timing of the light are synchronized.

In the operation method of the ultrasonic photoacoustic imaging apparatus of the present invention, it is preferable that a combined image of the ultrasonic image and the photoacoustic image is generated. In this case, it is preferable that the combined image is generated after matching scales of the ultrasonic image and the photoacoustic image.

Further, it is preferable that the first addition process is performed on a plurality of mixed signals on which a first frequency analysis process has been performed, and the second addition process is performed on a plurality of the mixed signal on which a second frequency analysis process has been performed, the second frequency analysis process being different from the first frequency analysis process in condition.

Still further, it is preferable that a third frequency analysis process is performed on an electrical signal generated by the first addition process, and a fourth frequency analysis process is performed on an electrical signal generated by the second addition process, the fourth frequency analysis process being different from the third frequency analysis process in condition.

In the ultrasonic photoacoustic imaging apparatus of the present invention, the acoustic image generation unit is configured to generate, based on a mixed signal obtained by converting an acoustic wave, in which an ultrasonic wave and a photoacoustic wave are mixed, detected by each transducer during a predetermined capturing period to an electrical signal, and making use of a difference between phase shift aspect of electrical signals of the ultrasonic waves from the same reflection source in the inside of a subject in the respective mixed signals and phase shift aspect of electrical signals of the photoacoustic waves from the same generation source in the inside of the subject in the respective mixed signals, an electrical signal reflecting the ultrasonic wave and an electrical signal reflecting the photoacoustic wave, and to generate an ultrasonic image based on the electrical signal reflecting the ultrasonic wave and a photoacoustic image based on the electrical signal reflecting the photoacoustic wave. Here, the difference between phase shift aspect of electrical signals of the ultrasonic waves and phase shift aspect of electrical signals of the photoacoustic waves arises from the difference in propagation distance between the ultrasonic waves and photoacoustic waves (i.e., for ultrasonic waves, total of path length from the ultrasonic wave projection unit to the reflection source and path length from the reflection source to the probe, while for photoacoustic waves, path length from the generation source to the probe) and does not depend on the frequencies of ultrasonic waves and photoacoustic waves. Consequently, it is possible to independently generate an ultrasonic image and a photoacoustic image without depending on the frequencies of the ultrasonic wave and photoacoustic wave even though they are detected simultaneously in the ultrasonic photoacoustic imaging apparatus that employs ultrasonic imaging and photoacoustic imaging.

### BRIEF DESCRIPTION OF THE DRAWINGS

FIG. 1 is a block diagram of a first embodiment of the ultrasonic photoacoustic imaging apparatus of the present invention.

FIG. 2 is a conceptual diagram, illustrating timing control for synchronizing projection timings of ultrasonic wave and light.

FIG. 3 is a schematic cross-sectional view of an array transducer having a plurality of transducers, illustrating area division.

FIG. 4 is a conceptual diagram, illustrating detection of mixed signals by transducers with respect to each area.

FIG. 5 is a conceptual diagram, illustrating a process of generating a tomographic image using all mixed signals detected by the array transducer.

FIG. 6 is a conceptual diagram, illustrating a difference between phase shift aspect of ultrasonic waves from the same reflection source and phase shift aspect of photoacoustic waves from the same generation source.

FIG. 7 is a block diagram of a second embodiment of the ultrasonic photoacoustic imaging apparatus of the present invention.

### DESCRIPTION OF THE PREFERRED EMBODIMENTS

Hereinafter, embodiments of the present invention will be described with reference to the accompanying drawings, but it should be appreciated that the present invention is not limited to the embodiments to be described herein below. Note that each component in the drawings is not necessarily drawn to scale in order to facilitate visual recognition.

### First Embodiment of Ultrasonic Photoacoustic Imaging Apparatus and Operation Method of the Same

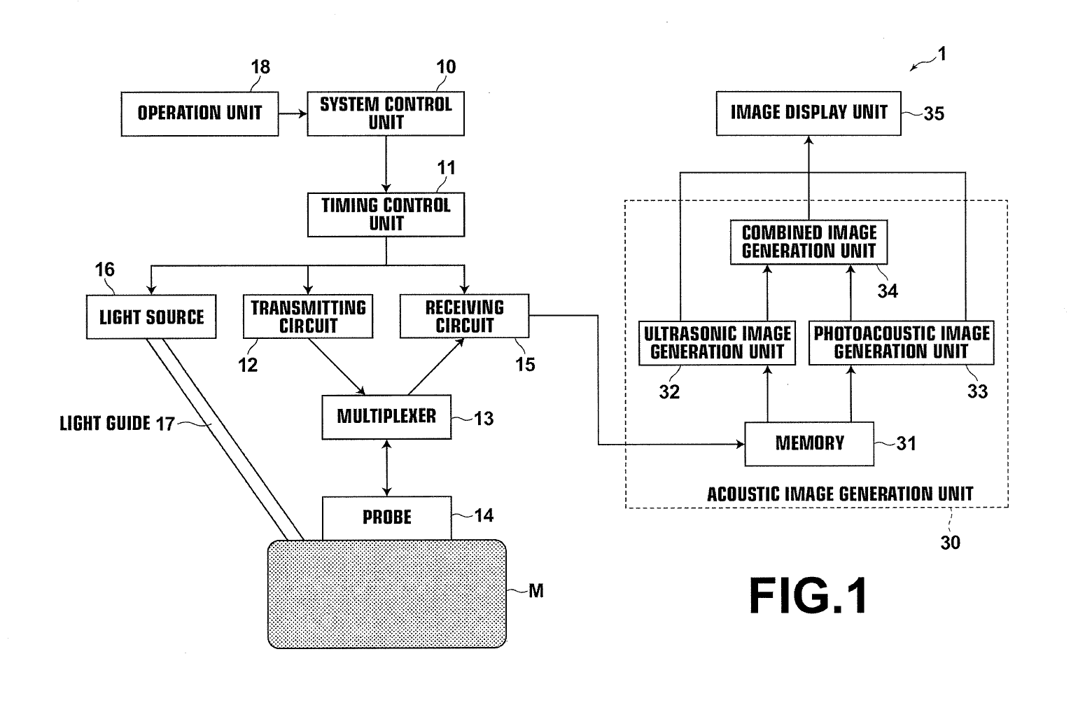

A first embodiment of the ultrasonic photoacoustic imaging apparatus and an operation method of the same will be described in detail. FIG. 1 is a block diagram of the ultrasonic photoacoustic imaging apparatus according to the first embodiment.

As illustrated in FIG. 1, ultrasonic photoacoustic imaging apparatus 1 of the present invention includes system control unit 10 for controlling the entire system, timing control unit 11 for controlling projection timings of an ultrasonic wave and light, as well as the timing of acoustic wave capturing period, transmitting circuit 12 for giving a predetermined delay time to a transmitting signal, multiplexer 13, and probe 14 which includes an array transducer having a plurality of transducers and is capable of projecting an ultrasonic wave into the inside of subject M and converting an acoustic wave propagating in the inside of subject M to an electrical signal. Ultrasonic photoacoustic imaging apparatus 1 further includes receiving circuit 15 for giving a predetermined delay time to a receiving signal, light source 16 for projecting light into the inside of subject M, light guide 17 for guiding light from light source 16 to subject M, operation unit 18 for use by the operator to set patient information and imaging conditions of the imaging apparatus, acoustic image generation unit 30 for generating an ultrasonic image, a photoacoustic image, and a combined image thereof based on a receiving signal of the acoustic wave detected by probe 14, and image display unit 35 for displaying a tomographic image generated by acoustic image generation unit 30. Here, acoustic image generation unit 30 generates, based on a mixed signal obtained by converting an acoustic wave, in which an ultrasonic wave and a photoacoustic wave are mixed, detected by each transducer during a predetermined capturing period to an electrical signal, and making use of a difference between phase shift aspect of electrical signals of the ultrasonic waves from the same reflection source in the inside of subject M in the respective mixed signals and phase shift aspect of electrical signals of the photoacoustic waves from the same generation source in the inside of subject M in the respective mixed signals, an electrical signal reflecting the ultrasonic wave and an electrical signal reflecting the photoacoustic wave, and generates an ultrasonic image based on the electrical signal reflecting the ultrasonic wave and a photoacoustic image based on the electrical signal reflecting the photoacoustic wave. In the present embodiment, light source 16 and light guide 17 function as the light projection unit of the present invention, and probe 14 doubles as the ultrasonic wave projection unit of the present invention.

An ultrasonic photoacoustic imaging apparatus operation method according to the first embodiment is a method including the steps of projecting an ultrasonic wave and light into an inside of subject M using the apparatus described above, using probe 14, detecting an ultrasonic wave reflected from the inside of subject M and converting the detected ultrasonic wave to an electrical signal and detecting a photoacoustic wave generated in the inside of subject M and converting the photoacoustic wave to an electrical signal, and generating an ultrasonic image based on the electrical signal of the detected ultrasonic wave and/or a photoacoustic image based on the electrical signal of the detected photoacoustic wave in which, based on a mixed signal obtained by converting an acoustic wave, in which an ultrasonic wave and a photoacoustic wave are mixed, detected by each transducer during a predetermined capturing period to an electrical signal, and making use of a difference between phase shift aspect of electrical signals of ultrasonic waves from the same reflection source in the inside of subject M in the respective mixed signals and phase shift aspect of electrical signals of photoacoustic waves from the same generation source in the inside of subject M in the respective mixed signals, an electrical signal reflecting the ultrasonic wave and an electrical signal reflecting the photoacoustic wave are generated, and an ultrasonic image is generated based on the electrical signal reflecting the ultrasonic wave and a photoacoustic image is generated based on the electrical signal reflecting the photoacoustic wave.

System control unit 10 includes, for example, a CPU, storage circuit, and the like, and controls each unit, such as timing control unit 11, transmitting circuit 12, receiving circuit 15, acoustic image generation unit 30, and the like, according to a command signal from the operation unit, as well as performing overall control of the system.

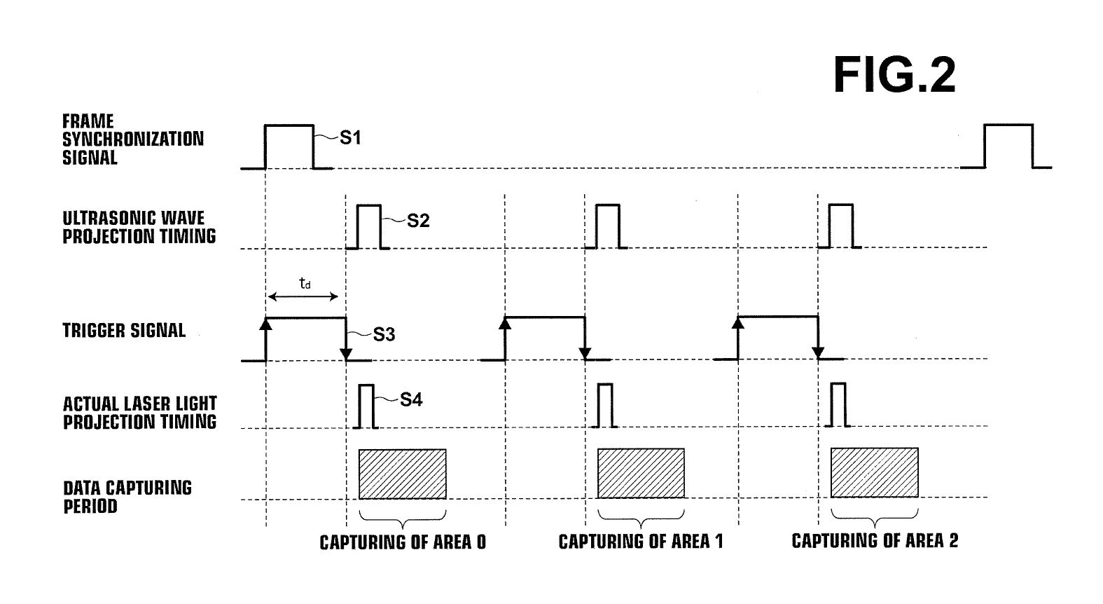

Timing control unit 11 controls the projection timings of ultrasonic wave and light, as well as the timing of acoustic wave capturing period. Considering that a combined image of an ultrasonic image and a photoacoustic image will be generated, it is preferable that ultrasonic image data and photoacoustic image data are collected without any time lag. If time lag exists between the two data collection periods, distortion is caused between the tomographic images by the motion of the subject during the time lag and image quality of the combined image is degraded. Consequently, timing control unit 11 performs control such that projection timing of the ultrasonic wave and projection timing of the light are synchronized. More specifically, the following control is performed. FIG. 2 is a conceptual diagram, illustrating timing control for synchronizing projection timings of ultrasonic wave and light. Collection of data required for generating one frame of an ultrasonic image and one frame of a photoacoustic image is initiated in synchronization with frame synchronization signal S1. First, timing control unit 11 generates a trigger signal S3 having a pulse width td and outputs the trigger signal S3 to transmitting circuit 12, receiving circuit 15, and light source 16. Here, note that td corresponds to a time lapse after the trigger signal S3 is received by light source 16 and before light is actually emitted from light source 16 (delay time to actual emission). By setting light source 16 to be driven in synchronization with the rising edge of the trigger signal S3, the actual light projection timing S4 is after the delay time td. In the mean time, transmitting circuit 12 has substantially no delay time with respect to the trigger signal S3. Therefore, transmitting circuit 12 is set to generate, in synchronization with the trailing edge of the trigger signal S3, a pulse with a pulse width corresponding to a transducer band and to output the pulse to the ultrasonic wave projection unit (probe). This results in that the ultrasonic wave projection timing S2 substantially coincides with the trailing edge of the trigger signal S3. This allows the ultrasonic wave projection timing S2 to be synchronized with the light projection timing S4. Then, receiving circuit 15 is set to perform data capture in synchronization with the trailing edge of the trigger signal S3, thereby allowing timing control reduced in time lag between the two data collection periods to be realized.

Transmitting circuit 12 includes a transmission delay circuit and a drive circuit. The transmission delay circuit may control the focus position of a transmission ultrasonic wave. The drive circuit generates a high voltage pulse (impulse with a crest value of several hundreds of volts) for driving the transducer and outputs the pulse to the transducer, whereby an ultrasonic wave may be generated.

Multiplexer 13 is designed to select n adjacent transducers from N transducers of the array transducer (n<N) when an ultrasonic wave is transmitted or received, or when a photoacoustic wave is received.

Probe 14 includes an array transducer having a plurality of transducers and designed to detect an acoustic wave (ultrasonic wave and/or photoacoustic wave) propagating in the inside of a subject M. In the present embodiment, the probe also has a function as the ultrasonic wave projection unit, but it is not necessarily required. The transducer is a piezoelectric device, such as a piezoelectric ceramics, a polymer film, e.g., polyvinylpyrrolidone fluoride, or the like.

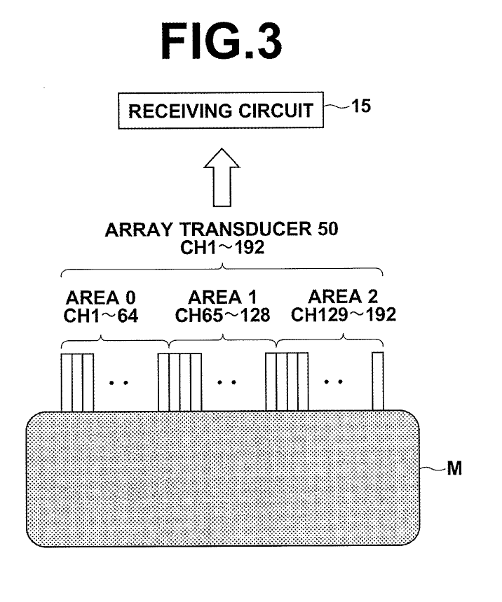

FIG. 3 illustrates example array transducer 50 having 192 transducers CH1 to CH192, in which array transducer 50 is handled by divided into three areas of area 0 (area of transducers CH1 to CH64), area 1 (area of transducers CH65 to CH128), and area 2 (area of transducers CH129 to CH192). If array transducer 50 having N transducers is handled as n (n<N) adjacent transducer groups (areas) and imaging operation is performed with respect to each area in the manner described above, not all of the channel transducers require a preamplifier or an A/D converter and thereby the structure of the probe may be simplified with reduced cost. Further, if a plurality of optical fibers is provided to individually project light onto the respective areas, optical power for one projection may be reduced, which provides an advantage that a high power, expensive light source is not required.

Preferably, ultrasonic wave projection is performed using a collimated ultrasonic wave in order not to cause intensity difference in the acoustic field of each area. If the focus position of the ultrasonic wave is set to not less than 100 mm, the ultrasonic wave to be projected can be regarded as a substantially collimated wave because the photoacoustic imaging range is generally about 40 mm.

Receiving circuit 15 includes a preamplifier and an A/D converter. The preamplifier amplifies a small electrical signal received by a transducer selected by the multiplexer, thereby ensuring a sufficient S/N ratio. The electrical signal ensured sufficient S/N ratio by the preamplifier is converted to a digital signal by the A/D converter and the digital signal is stored in a memory.

As for light source 16, a semiconductor laser, a light emitting diode, a solid-state laser, or the like may be used. Preferably, light source 16 emits, as the light, a pulse light having a pulse width of 1 to 100 nsec. The wavelength of the light is determined as appropriate according to the light absorption characteristics of the measurement target substance within a subject. For example, when the measurement target substance is hemoglobin in a living body, a wavelength of 600 to 1000 nm is preferably used. Further, it is preferable that the wavelength of the light is in the range from 700 to 1000 nm from the viewpoint that such light can reach a deep portion of a subject M. Preferably, the power of the light is in the range from 10 μJ/cm² to 10 mJ/cm² from the viewpoint of propagation losses of the light and photoacoustic wave, conversion efficiency to the photoacoustic wave, detection sensitivity of current detectors, and the like. Preferably, the repetition of the pulse light projection is 10 Hz or more from the viewpoint of image construction speed. Further, the measuring light may also be a pulse string in which a plurality of the pulse light is arranged.

Light guide 17 is provided to guide the light emitted from light source 16 to a subject M and an optical fiber is preferably used in order to efficiently guide the light. Light guide 17 may be provided in a plurality in order to perform uniform light projection. Although not clearly shown in FIG. 1, light guide 17 may be used in combination with an optical system, such as an optical filter, a lens, and the like.

Operation unit 18 includes an operation screen, a keyboard, a mouse, and the like, and is used by the operator to set necessary information, such as patient information, imaging conditions, and the like, to apparatus 1.

Acoustic image generation unit 30 is a section for generating an ultrasonic image and/or a photoacoustic image based on electrical signals of ultrasonic wave and photoacoustic wave detected by the probe, as well as a combined image thereof. For this purpose, it includes memory 31 for storing a mixed signal detected by the probe, ultrasonic image generation unit 32, photoacoustic image generation unit 33, and combined image generation unit 34 for generating a combined image using the generated ultrasonic image and photoacoustic image.

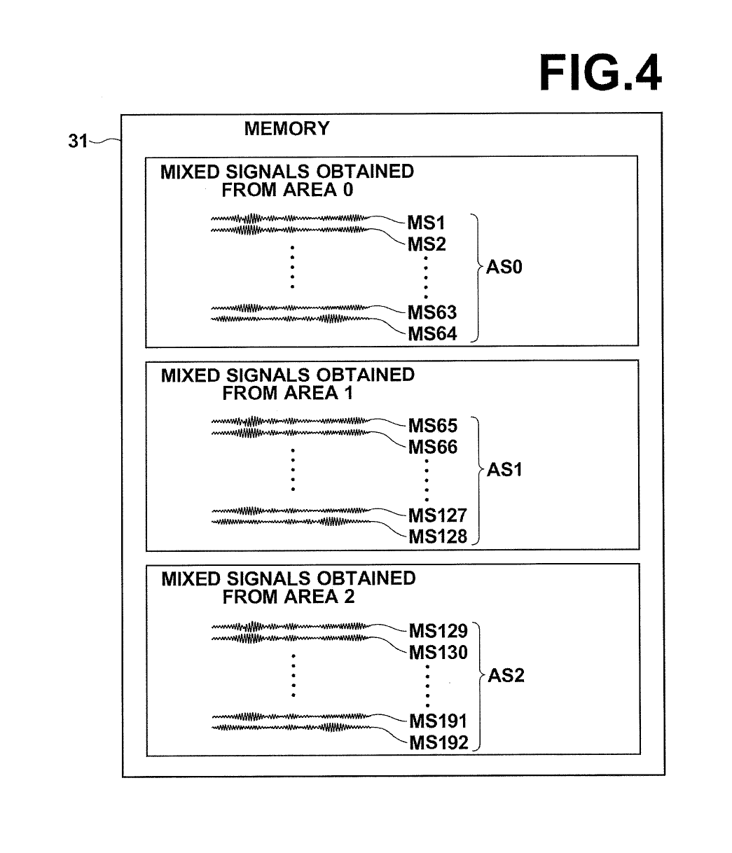

Memory 31 is an area for storing a mixed signal detected by each transducer of the array transducer. FIG. 4 is a conceptual diagram, illustrating the state in which mixed signals MS1 to MS192 detected by each of transducers CH1 to CH192 of the 192 channel array transducer through imaging operation with respect to each area are grouped with respect to each of the areas (AS0 to AS2) and stored.

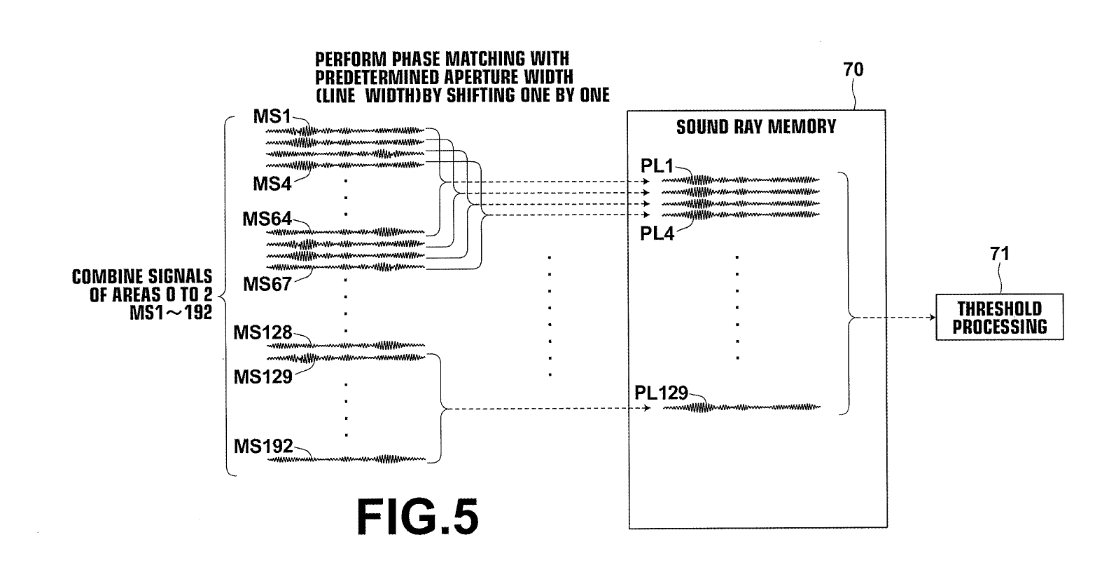

Ultrasonic image generation unit 32 and photoacoustic image generation unit 33 generate an ultrasonic image and a photoacoustic image respectively based on the mixed signals MS1 to MS192 stored in the memory. For example, the photoacoustic image is generated from the mixed signals in the following manner. First, the entire information of mixed signals AS0 to AS2 grouped with respect to each area and stored is combined together as one unit and phase matching is performed on the combined signal with a predetermined aperture width (line width) by shifting one by one and one line of photoacoustic image corresponding to the aperture width is obtained. FIG. 5 is a conceptual diagram illustrating phase matching performed when an array transducer having 192 transducers is used. More specifically, mixed signals MS1 to MS64 detected by the transducers CH1 to CH64 are set as a predetermined aperture width and one line of photoacoustic image PL1 with respect to the aperture width is obtained. Then the channels are shifted by one and mixed signals MS2 to MS65 detected by the transducers CH2 to CH65 are set as a predetermined aperture width and one line of photoacoustic image PL2 with respect to the aperture width is obtained. Then, such operation is repeated until one line of photoacoustic image PL129 is obtained by setting mixed signals MS128 to MS192 as the predetermined aperture width. In this way, line data necessary to generate a photoacoustic image are generated. A plurality of one line photoacoustic images PL1 to PL129 obtained in the manner described above is stored in sound ray memory 70 and subjected to required signal processing, such as threshold processing 71, to be described later. Then, one frame of the photoacoustic image is generated by combining the plurality of one line photoacoustic images PL1 to PL129 and the generated image is outputted to image display unit 35 or combined image generation unit 34.

Here, the description has been made of a case in which a photoacoustic image is generated from mixed signals. But an ultrasonic image can also be generated from the same mixed signals in the similar manner except for the phase matching condition, in which a plurality of one line ultrasonic images is obtained, then one frame of the ultrasonic image is generated by combining the plurality of one line ultrasonic images and the generated image is outputted to image display unit 35 or combined image generation unit 34. Preferably, image generation in ultrasonic image generation unit 32 and image generation in photoacoustic image generation unit 33 are performed in parallel from the viewpoint of improving image construction speed. This is a further advantage that can be realized by the advantageous effect of the present invention that “even though an ultrasonic wave and a photoacoustic wave are detected simultaneously, an ultrasonic image and a photoacoustic image can be generated independently without depending on the frequencies thereof”.

Note that each of ultrasonic image generation unit 32 and photoacoustic image generation unit 33 may have a frequency filter on the input side, on the output side, or on each side. That is, a frequency filter is provided on the input side of ultrasonic image generation unit 32 to perform a first frequency analysis process and a first addition process is performed on a plurality of mixed signals on which the first frequency analysis process has been performed, while a frequency filter is provided on the input side of photoacoustic image generation unit 33 to perform a second frequency analysis process which is different in condition from the first frequency analysis process and a second addition process is performed on a plurality of mixed signals on which the second frequency analysis process has been performed. In this case, the first frequency analysis process and second frequency analysis process may perform filtering for different frequencies according to the difference in frequency between the ultrasonic wave and photoacoustic wave. For example, the pulse length of the light may be adjusted such that an ultrasonic wave with a frequency of 5 to 8 MHz is detected while a photoacoustic wave with a frequency of about 3 MHz is detected. Further, a frequency filter is provided on the output side of ultrasonic image generation unit 32 to perform a third frequency analysis process on the electrical signal generated by the first addition process, while a frequency filter is provided on the output side of photoacoustic image generation unit 33 to perform a fourth frequency analysis process which is different in condition from the third frequency analysis process. In this case, the third frequency analysis process and fourth frequency analysis process may perform filtering for different frequencies according to the difference in frequency between the ultrasonic wave and photoacoustic wave. These may further prevent interference between the ultrasonic wave and photoacoustic wave. Further, it is also possible to combine all of the first to fourth frequency analysis processes.

Next, phase matching conditions will be described. Ultrasonic photoacoustic imaging apparatus 1 of the present invention features that acoustic image generation unit 30 is configured to generate, based on a mixed signal obtained by converting an acoustic wave, in which an ultrasonic wave and a photoacoustic wave are mixed, detected by each transducer during a predetermined capturing period to an electrical signal, and making use of a difference between phase shift aspect of electrical signals of the ultrasonic waves from the same reflection source in the inside of subject M in the respective mixed signals and phase shift aspect of electrical signals of the photoacoustic waves from the same generation source in the inside of subject M in the respective mixed signals, an electrical signal reflecting the ultrasonic wave and an electrical signal reflecting the photoacoustic wave, and to generate an ultrasonic image based on the electrical signal reflecting the ultrasonic wave and a photoacoustic image based on the electrical signal reflecting the photoacoustic wave.

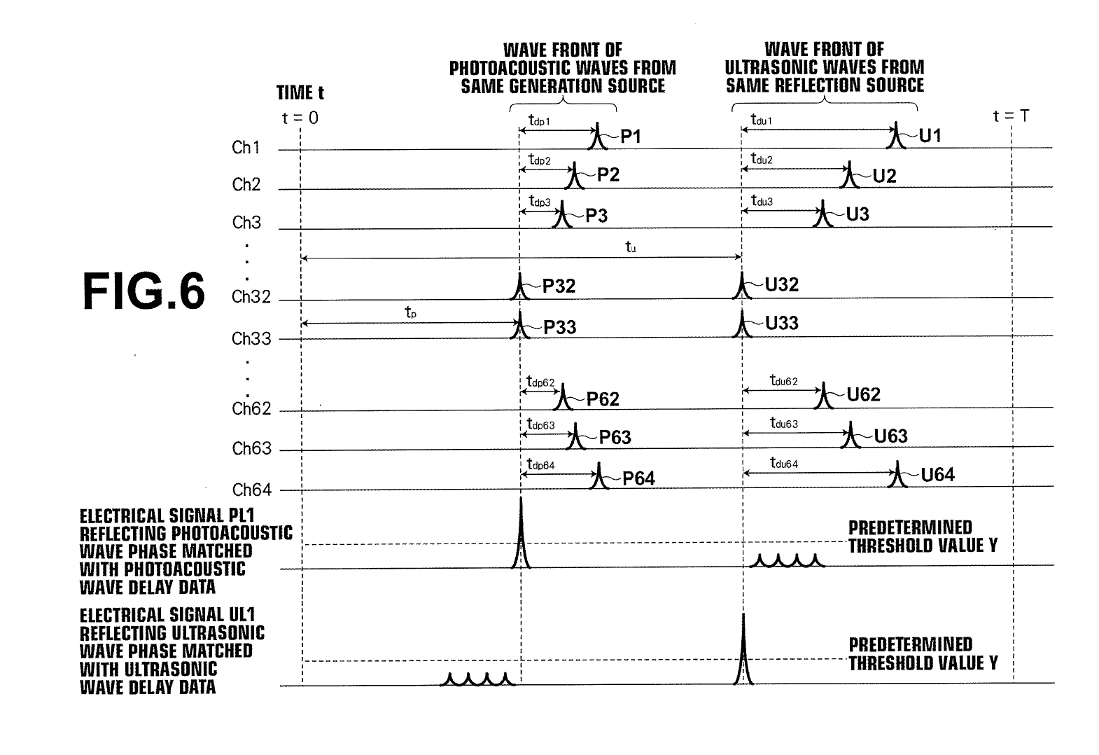

FIG. 6 shows, by way of example, measurement of area 0 by projecting an ultrasonic wave and light simultaneously, and illustrates a difference between phase shift aspect of ultrasonic waves from the same reflection source of a subject and phase shift aspect of photoacoustic waves from the same generation source of the subject.

First, in a case where light is projected (t=0), it may be deemed that the light reaches the measurement target region of the subject as soon as the light is projected because the propagation speed of the light is sufficiently faster than that of the ultrasonic wave. The projection of the light induces a photoacoustic effect and a photoacoustic wave is generated. The generated photoacoustic wave propagates as a spherical wave from the generation source and reaches the array transducer (probe). At this time, due to the positional relationship between each transducer of the array transducer and the generation source of the photoacoustic wave, the propagation distance of the photoacoustic wave from the generation source to each transducer is different. Thus, a phase shift corresponding to a difference in the propagation distance occurs between each photoacoustic wave from the same generation source detected by each transducer.

In the mean time, in a case where an ultrasonic wave is projected (t=0), an ultrasonic wave projected from each transducer of the array transducer propagates a reciprocating path with respect to a reflection source in the subject. At this time, due to the positional relationship between each transducer of the array transducer and the reflection source of the ultrasonic wave, the propagation distance of the ultrasonic wave is different. Thus, a phase shift corresponding to a difference in the propagation distance occurs as in the photoacoustic wave. In addition, phase shift aspect with respect to the ultrasonic wave differs from phase shift aspect with respect to the photoacoustic wave because, unlike the photoacoustic wave, the ultrasonic wave propagates through a reciprocating path.

The term “phase shift aspect” as used herein refers to a time difference between the time when an ultrasonic wave from the same reflection source (or a photoacoustic wave from the same generation source) is detected by a reference transducer (e.g., transducer CH 32 or CH 33 in FIG. 6) and the time when an ultrasonic wave from the same reflection source (or a photoacoustic wave from the same generation source) is detected by each of the other transducers. In other words, it can be said to be a curvature of the wave front when an ultrasonic wave from the same reflection source (or a photoacoustic wave from the same generation source) is detected by the array transducer. The term “difference in phase shift aspect” as used herein refers to that the time difference aspect (or the curvature of the wave front) as a whole does not correspond to each other between the ultrasonic wave and photoacoustic wave.

As described above, there is a difference in phase shift aspect between the ultrasonic wave and photoacoustic wave. This implies that different amounts of delay are applied to the ultrasonic wave and photoacoustic wave when phase matching is performed. Consequently, the use of the difference in phase shift aspect allows, even though an acoustic wave, in which an ultrasonic wave and a photoacoustic wave are mixed, is detected simultaneously by the same detector (probe), an ultrasonic image and photoacoustic image to be generated independently without depending on the frequencies thereof.

For example, with respect to photoacoustic waves P1 to P64 from the same generation source detected by transducers CH1 to CH64 in FIG. 6, amounts of delay of the photoacoustic waves P1 to P31 and P34 to P64 with respect to the reference photoacoustic waves P32 and P33 are t_{dp1} to t_{dp31} and t_{dp34} to t_{dp64}. These amounts of delay t_{dp1} to t_{dp31} and t_{dp34} to t_{dp64} are the values that can be determined by the geometrical positional relationship between the array transducer (transducers in area 0 in this case) and generation source, i.e., the depth of the generation source from the surface. Here, the depth may be derived from the time between the time when the light is projected and the time when reference photoacoustic waves P32 and P33 are detected, i.e., tₚ in FIG. 6. Therefore, the signal strength of a phase matched photoacoustic wave at certain time t may be obtained by Formula (1) given below (second addition process).

ΣCHi(t+t_{dpi}) (1)

where, Σ is a total sum with respect to i, i represents an integer from 1 to 64, and CHi(t) is a signal strength of i^{th} transducer CHi at time t.

Using Formula (1), calculations are made from time t=0 to t=T, and signal strength values are taken on vertical axis with the horizontal axis representing time t to obtain an electrical signal PL1 reflecting the photoacoustic waves in FIG. 6. In the electrical signal PL1, the signal strength at t=tₚ is amplified as the result of addition through phase matching of the photoacoustic waves, while the signal strength at t=tu is not amplified since the phase matching condition is improper for the ultrasonic waves. That is, by making use of a difference in phase shift aspect between the ultrasonic wave and photoacoustic wave, it is understood that the electrical signal PL1 reflecting the photoacoustic waves can be obtained based on a plurality of mixed signals. There may be a case in which influence of signal strength of an ultrasonic wave can not be completely eliminated, as in the electrical signal PTA in FIG. 6. In such a case, threshold processing in which signal strength less than a predetermined threshold value Y is decreased (second threshold process) may be performed and thereby contrast of the photoacoustic wave may be improved. Although the threshold value Y may be set as appropriate, it is preferable that the value is zero from the viewpoint of maximizing the contrast of the photoacoustic wave.

Then, electrical signals PL1 to PL129 reflecting photoacoustic waves obtained through processing identical to that described above are combined to generate a photoacoustic image.

An ultrasonic image may also be generated through processing identical to that described above. That is, in FIG. 6, amounts of delay of the ultrasonic waves U1 to U31 and U34 to U64 with respect to the reference ultrasonic waves U32 and U33 are t_{du1} to t_{du31} and t_{du34} to t_{du64}. These amounts of delay t_{du1} to t_{du31} and t_{du34} to t_{du64} are the values that can be determined by the geometrical positional relationship between the array transducer (transducers in area 0 in this case) and reflection source, i.e., the depth of the reflection source from the surface. Here, the depth may be derived from the time between the time when the ultrasonic wave is projected and the time when reference ultrasonic waves U32 and U33 are detected, i.e., tᵤ in FIG. 6. Therefore, the signal strength of a phase matched ultrasonic wave at certain time t may be obtained by Formula (2) given below (first addition process).

ΣCHi(t+t_{dui}) (2)

where, Σ, i, and CHi are the same as those in Formula (1) above.

Using Formula (2), calculations are made from time t=0 to t=T, and signal strength values are taken on vertical axis with the horizontal axis representing time t to obtain an electrical signal UL1 reflecting the ultrasonic waves in FIG. 6. In the electrical signal UL1, the signal strength at t=tᵤ is amplified as the result of addition through phase matching of the ultrasonic waves, while the signal strength at t=tp is not amplified since the phase matching condition is improper for the photoacoustic waves. That is, by making use of a difference in phase shift aspect between the ultrasonic wave and photoacoustic wave, it is understood that the electrical signal UL1 reflecting the ultrasonic waves can be obtained based on a plurality of mixed signals. There may be a case in which influence of signal strength of a photoacoustic wave can not be completely eliminated, as in the electrical signal UL1 in FIG. 6. In such a case, threshold processing in which signal strength less than a predetermined threshold value Y is decreased (first threshold process) may be performed and thereby contrast of the ultrasonic wave may be improved. Although the threshold value Y may be set as appropriate, it is preferable that the value is zero from the viewpoint of maximizing the contrast of the ultrasonic wave. Further, the threshold values of the first and second threshold processes are not necessarily the same.

Then, electrical signals UL1 to UL129 reflecting ultrasonic waves obtained through processing identical to that described above are combined to generate an ultrasonic image.

Combined image generation unit 34 generates a combined image by superimposing the ultrasonic image and photoacoustic image obtained in the manner described above. Here, it is possible that the ultrasonic image and photoacoustic image may be superimposed in an identifiable manner, for example, displaying the ultrasonic image in monochrome while the photoacoustic image in red. As can be seen from the electrical signal UL1 reflecting ultrasonic waves and the electrical signal PL1 reflecting photoacoustic waves in FIG. 6, the propagation distance of the ultrasonic wave is longer than that of the photoacoustic wave. This produces a combined image, if the electrical signals UL1 and PL1 are directly superimposed, that appears as if acoustic waves were measured from independent two regions when the same region is actually measured as the reflection source and generation source. Therefore, it is preferable that scale matching is performed on either one or both of the images.

As described above, in the ultrasonic photoacoustic imaging apparatus of the present invention, the acoustic image generation unit is configured to generate, based on a mixed signal obtained by converting an acoustic wave, in which an ultrasonic wave and a photoacoustic wave are mixed, detected by each transducer during a predetermined capturing period to an electrical signal, and making use of a difference between phase shift aspect of electrical signals of the ultrasonic waves from the same reflection source in the inside of a subject in the respective mixed signals and phase shift aspect of electrical signals of the photoacoustic waves from the same generation source in the inside of the subject in the respective mixed signals, an electrical signal reflecting the ultrasonic wave and an electrical signal reflecting the photoacoustic wave, and to generate an ultrasonic image based on the electrical signal reflecting the ultrasonic wave and a photoacoustic image based on the electrical signal reflecting the photoacoustic wave. Here, the difference in phase shift aspect between electrical signals of the ultrasonic wave and photoacoustic wave occurs due to a difference in propagation distance between the ultrasonic wave and photoacoustic wave. That is, the difference in phase shift aspect between electrical signals of the ultrasonic wave and photoacoustic wave does not depend on the frequencies thereof. As a result, in the ultrasonic photoacoustic imaging apparatus that uses ultrasonic imaging and photoacoustic imaging, even though the ultrasonic wave and photoacoustic wave are detected simultaneously, an ultrasonic image and a photoacoustic image may be generated independently without depending on the frequencies thereof.

### Second Embodiment of Ultrasonic Photoacoustic Imaging Apparatus and Operation Method of the Same

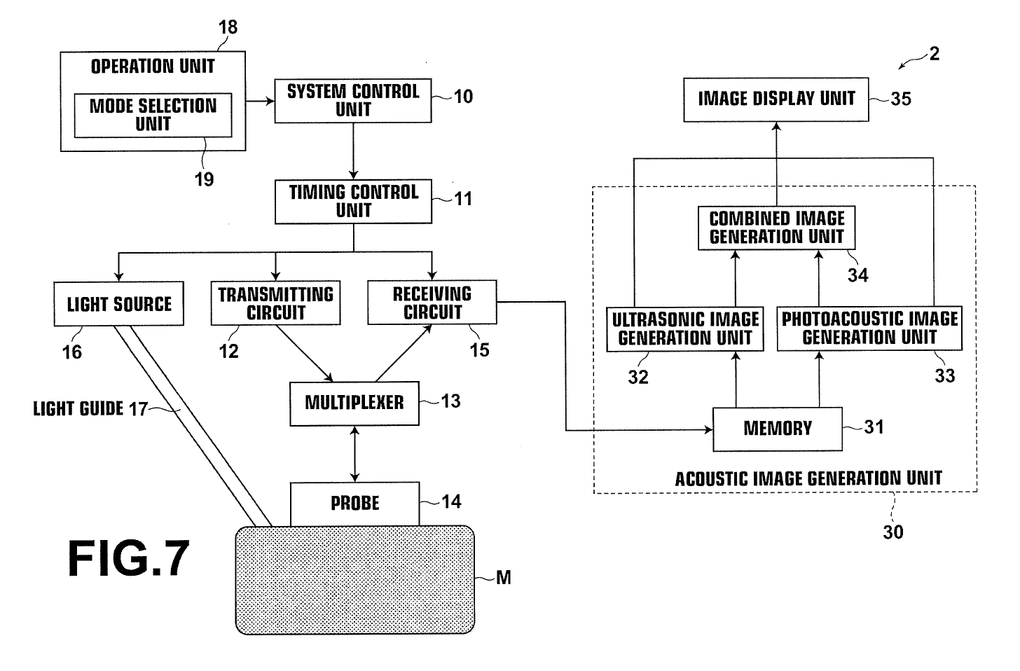

A second embodiment of the ultrasonic photoacoustic imaging apparatus and operation method of the same will now be described in detail. FIG. 7 is a block diagram of the ultrasonic photoacoustic imaging apparatus according to the second embodiment. Second ultrasonic photoacoustic imaging apparatus 2 and operation method of the same are similar to those of the first embodiment. The second embodiment differs from the first embodiment in that operation unit 18 includes mode selection unit 19. Therefore, description will be made focusing on mode selection unit 19 and other components will not be elaborated upon further here unless otherwise specifically required.

Mode selection unit 19 allows selection between an ultrasonic mode in which only the ultrasonic image is generated and a photoacoustic mode in which only a photoacoustic image is generated. Further, mode selection unit 19 allows switching between projection and non-projection of the ultrasonic wave in the photoacoustic mode. Through mode selection unit 19, the operator may confirm only a conventional ultrasonic image as required or may confirm influence of interference between the ultrasonic wave and photoacoustic wave on the spot through comparison by switching between the projection and non-projection of the ultrasonic wave.

As described above, also in the ultrasonic photoacoustic imaging apparatus of the present invention, the acoustic image generation unit is configured to generate, based on a mixed signal obtained by converting an acoustic wave, in which an ultrasonic wave and a photoacoustic wave are mixed, detected by each transducer during a predetermined capturing period to an electrical signal, and making use of a difference between phase shift aspect of electrical signals of the ultrasonic waves from the same reflection source in the inside of a subject in the respective mixed signals and phase shift aspect of electrical signals of the photoacoustic waves from the same generation source in the inside of the subject in the respective mixed signals, an electrical signal reflecting the ultrasonic wave and an electrical signal reflecting the photoacoustic wave, and to generate an ultrasonic image based on the electrical signal reflecting the ultrasonic wave and a photoacoustic image based on the electrical signal reflecting the photoacoustic wave. Therefore, advantageous effects identical to those of the first embodiment may be obtained.

What is claimed is:

1. An ultrasonic photoacoustic imaging apparatus, comprising:
an ultrasonic wave projection unit for projecting an ultrasonic wave into an inside of a subject;
a light projection unit for projecting light into the inside of the subject;
a probe capable of detecting the ultrasonic wave reflected from the inside of the subject by the projection of the ultrasonic wave into the inside of the subject and converting the detected ultrasonic wave into an electrical signal, and capable of detecting a photoacoustic wave generated in the inside of the subject by the projection of the light into the inside of the subject and converting the detected photoacoustic wave into an electrical signal; and
an acoustic image generation unit for generating an ultrasonic image based on the electrical signal of the ultrasonic wave detected by the probe and/or a photoacoustic image based on the electrical signal of the photoacoustic wave detected by the probe, wherein:
the probe includes an array transducer having a plurality of transducers; and
the acoustic image generation unit is a unit capable of generating, based on a mixed signal obtained by converting an acoustic wave, in which an ultrasonic wave and a photoacoustic wave are mixed, detected by each transducer during a predetermined capturing period to an electrical signal, and making use of a difference between phase shift aspect of electrical signals of the ultrasonic waves from the same reflection source in the inside of the subject in the respective mixed signals and phase shift aspect of electrical signals of the photoacoustic waves from the same generation source in the inside of the subject in the respective mixed signals, an electrical signal reflecting the ultrasonic wave and an electrical signal reflecting the photoacoustic wave, and generating the ultrasonic image based on the electrical signal reflecting the ultrasonic wave and the photoacoustic image based on the electrical signal reflecting the photoacoustic wave.

2. The ultrasonic photoacoustic imaging apparatus of claim 1, wherein the acoustic image generation unit is a unit that generates the electrical signal reflecting the ultrasonic wave by performing a first addition process for adding a plurality of mixed signals using ultrasonic wave delay data and under the condition of matching the phase shift of the electrical signal of the ultrasonic wave, and generates the electrical signal reflecting the photoacoustic wave by performing a second addition process for adding a plurality of mixed signals using photoacoustic wave delay data and under the condition of matching the phase shift of the electrical signal of the photoacoustic wave.

3. The ultrasonic photoacoustic imaging apparatus of claim 2, wherein the acoustic image generation unit is a unit that generates the electrical signal reflecting the ultrasonic wave by performing a first threshold process on an electrical signal generated by the first addition process for decreasing signal strength less than a predetermined threshold value and generates the electrical signal reflecting the photoacoustic wave by performing a second threshold process on an electrical signal generated by the second addition process for decreasing signal strength less than a predetermined threshold value.

4. The ultrasonic photoacoustic imaging apparatus of claim 1, wherein the ultrasonic wave projection unit is a unit that projects a collimated ultrasonic wave.

5. The ultrasonic photoacoustic imaging apparatus of claim 4, further comprising a timing control unit for performing control such that projection timing of the collimated ultrasonic wave and projection timing of the light are synchronized.

6. The ultrasonic photoacoustic imaging apparatus of claim 1, wherein the acoustic image generation unit is a unit that generates the ultrasonic image and the photoacoustic image in parallel.

7. The ultrasonic photoacoustic imaging apparatus of claim 1, wherein the acoustic image generation unit is a unit that generates a combined image of the ultrasonic image and the photoacoustic image.

8. The ultrasonic photoacoustic imaging apparatus of claim 7, wherein the acoustic image generation unit is a unit that generates the combined image after matching scales of the ultrasonic image and the photoacoustic image.

9. The ultrasonic photoacoustic imaging apparatus of claim 1, wherein the apparatus allows selection between an ultrasonic mode in which only the ultrasonic image is generated and a photoacoustic mode in which the photoacoustic is generated.

10. The ultrasonic photoacoustic imaging apparatus of claim 9, wherein the apparatus allows switching between projection and non-projection of the ultrasonic wave in the photoacoustic mode.

11. The ultrasonic photoacoustic imaging apparatus of claim 2, wherein the acoustic image generation unit is a unit that performs the first addition process on a plurality of mixed signals on which a first frequency analysis process has been performed and the second addition process on a plurality of mixed signal on which a second frequency analysis process has been performed, the second frequency analysis process being different from the first frequency analysis process in condition.

12. The ultrasonic photoacoustic imaging apparatus of claim 2, wherein the acoustic image generation unit is a unit that performs a third frequency analysis process on an electrical signal generated by the first addition process and a fourth frequency analysis process on an electrical signal generated by the second addition process, the fourth frequency analysis process being different from the third frequency analysis process in condition.

13. The ultrasonic photoacoustic imaging apparatus of claim 11, wherein the acoustic image generation unit is a unit that performs a third frequency analysis process on an electrical signal generated by the first addition process and a fourth frequency analysis process on an electrical signal generated by the second addition process, the fourth frequency analysis process being different from the third frequency analysis process in condition.

14. An ultrasonic photoacoustic imaging method comprising the steps of:
projecting an ultrasonic wave and light into an inside of a subject;
using a probe, detecting the ultrasonic wave reflected from the inside of the subject and converting the detected ultrasonic wave into an electrical signal, and detecting a photoacoustic wave generated in the inside of the subject and converting the detected photoacoustic wave into an electrical signal;
generating an ultrasonic image based on the electrical signal of the detected ultrasonic wave and/or a photoacoustic image based on the electrical signal of the detected photoacoustic wave, wherein:
the probe includes an array transducer having a plurality of transducers; and
based on a mixed signal obtained by converting an acoustic wave, in which an ultrasonic wave and a photoacoustic wave are mixed, detected by each transducer during a predetermined capturing period to an electrical signal, and making use of a difference between phase shift aspect of electrical signals of the ultrasonic waves from the same reflection source in the inside of the subject in the respective mixed signals and phase shift aspect of electrical signals of the photoacoustic waves from the same generation source in the inside of the subject in the respective mixed signals, an electrical signal reflecting the ultrasonic wave and an electrical signal reflecting the photoacoustic wave are generated; and
the ultrasonic image is generated based on the electrical signal reflecting the ultrasonic wave and the photoacoustic image is generated based on the electrical signal reflecting the photoacoustic wave.

15. The ultrasonic photoacoustic imaging method of claim 14, wherein:
the electrical signal reflecting the ultrasonic wave is generated by performing a first addition process for adding a plurality of mixed signals using ultrasonic wave delay data and under the condition of matching the phase shift of the electrical signal of the ultrasonic wave; and
the electrical signal reflecting the photoacoustic wave is generated by performing a second addition process for adding a plurality of mixed signals using photoacoustic wave delay data and under the condition of matching the phase shift of the electrical signal of the photoacoustic wave.

16. The ultrasonic photoacoustic imaging method of claim 15, wherein:
the electrical signal reflecting the ultrasonic wave is generated by performing a first threshold process on an electrical signal generated by the first addition process for decreasing signal strength less than a predetermined threshold value; and
the electrical signal reflecting the photoacoustic wave is generated by performing a second threshold process on an electrical signal generated by the second addition process for decreasing signal strength less than a predetermined threshold value.

17. The ultrasonic photoacoustic imaging method of claim 14, wherein a collimated ultrasonic wave is projected.

18. The ultrasonic photoacoustic imaging method of claim 17, wherein control is performed such that projection timing of the collimated ultrasonic wave and projection timing of the light are synchronized.

19. The ultrasonic photoacoustic imaging method of claim 15, wherein:
the first addition process is performed on a plurality of mixed signals on which a first frequency analysis process has been performed; and
the second addition process is performed on a plurality of the mixed signal on which a second frequency analysis process has been performed, the second frequency analysis process being different from the first frequency analysis process in condition.

20. The ultrasonic photoacoustic imaging method of claim 15, wherein:
a third frequency analysis process is performed on an electrical signal generated by the first addition process; and
a fourth frequency analysis process is performed on an electrical signal generated by the second addition process, the fourth frequency analysis process being different from the third frequency analysis process in condition.
